(19) United States
(12) Patent Application Publication
(10) Pub. No.: US 2022/0273855 A1
(43) Pub. Date: Sep. 1, 2022

(54) DEVICE AND METHOD FOR PREPARING A DONOR ORGAN FOR TRANSPLANTATION

(71) Applicant: SeaStar Medical, Inc., Denver, CO (US)

(72) Inventors: Charles Jack Fisher, Cardiff-By-The-Sea, CA (US); Christopher Jaynes, Centennial, CO (US); James R. Matson, Westminster, CO (US)

(21) Appl. No.: 17/597,983

(22) PCT Filed: Jul. 31, 2020
(86) PCT No.: PCT/US2020/044413

§ 371 Date: Jan. 31, 2022

Related U.S. Application Data
(60) Provisional application No. 62/881,695, filed on Aug. 1, 2019.

(51) Int. Cl. A61M 1/36; A61M 1/34; B01D 61/14; B01D 69/08; B01D 71/68

(52) U.S. Cl. CPC A61M 1/3621; A61M 1/34; B01D 61/145; B01D 69/081; B01D 71/68; A61M 2202/0413; A61M 2205/126; A61M 2205/3331; B01D 63/02

(57) ABSTRACT

The invention relates generally to methods of preparing one or more organs from a donor for transplantation to a recipient comprising passing blood from the donor through an extracorporeal membrane having a plurality of pores having an average pore size of at least 60 kDa.

### CROSS-REFERENCE TO RELATED APPLICATION

This application claims priority to and the benefit of U.S. Provisional Patent Application No. 62/881,695, filed on Aug. 1, 2019, the contents of which are incorporated by reference herein in their entirety.

BACKGROUND

In the United States, there is a critical shortage of donor organs. According to the U.S. Government, over 113,000 American men, women, and children were on the national transplant waiting list at the end of 2019, with twenty people dying each day while awaiting a suitable transplant. However, only 36,528 organ transplants were performed in 2018, with 10,722 deceased donors donating 29,697 organs. The remainder came from living donors (e.g., kidney donors).

While a deceased individual may have the potential to donate up to 7 solid organs (2 kidneys, 2 lungs, heart, liver, pancreas), along with intestines, many organs procured for donation are ultimately not transplanted into recipients due to the poor quality (for example, loss of function) of the organ. Further, many deceased individuals, despite donor status, are deemed ineligible for organ donation due to their cause of death or other health conditions. Brain death is associated with dramatic and serious pathophysiologic changes that adversely affect both the quantity and quality of organs available for transplant. Brain death creates a variety of inflammatory, hemodynamic and endocrine effects, which induce adverse sequelae in distant organs, leading to their degradation and ultimate unsuitability for transplantation. In particular, brain-death results in escalating gut/intestinal permeability, causing bacteria and toxins, such as endotoxins, to leak into the blood stream, stimulating a systemic increase in the production of pro-inflammatory cytokines and molecules. The resulting pro-inflammatory “cytokine storm” has damaging inflammatory effects throughout the body and leads to degradation of organs and tissues. These pro-inflammatory effects contribute to the fact that not all organs are available for procurement from a potential donor and not all procured organs are ultimately transplanted into a recipient.

There has been no change in the number of recovered and transplanted organs per deceased donor in over twenty years, with the average number of around 3.3 donor organs procured from brain-dead individuals. However, the U.S. Department of Health and Human Services has a goal of increasing that average to 3.75. In order to achieve this goal, there is a need for treatments and methods to not only increase the number of organs that can be procured from deceased individuals, but also for methods and treatments to improve the quality of organs procured.

### SUMMARY OF THE INVENTION

The invention is based, in part, upon the discovery that organ recovery and transplant outcomes, e.g., the number organs recovered and/or transplanted per donor and the quality of those organs, can be improved by passing blood from a donor, e.g., a brain-dead donor, through a semi-permeable membrane, for example, one or more membranes disposed in a hemofilter. Without wishing to be bound by theory, it is contemplated that processing the donor blood using this approach removes proinflammatory cytokines or other proinflammatory molecules from the blood, including microbes or cell-wall fragments or products of microbes (such as toxins) entering the blood stream from the gut, that negatively impact organ function, recovery and transplantation.

Accordingly, in one aspect, the invention provides a method of preparing one or more organs from a donor for transplantation to a recipient. In one embodiment, the method involves contacting blood from the donor with an extracorporeal membrane. The extracorporeal membrane defines a plurality of pores having an average pore size of at least 60 kDa. The pores permit proinflammatory molecules in the blood to pass therethrough for removal from the blood. The blood depleted of proinflammatory molecules is then returned to the donor. In some embodiments, the pores are defined by a wall (e.g., an inner wall or an outer wall) of a semi-permeable hollow fiber. In another embodiment, the membrane comprises a polymer.

In another aspect, the method involves passing blood from the donor through an extracorporeal cartridge comprising a housing and a plurality of semi-permeable hollow fibers disposed therein. Each of the semi-permeable hollow fibers comprises a lumen and a plurality of pores such that when the blood passes through the lumens of the hollow fibers, proinflammatory molecules from the blood pass through the pores and are removed from the blood. The blood depleted of proinflammatory molecules is then returned to the donor. The blood from the donor is passed through the cartridge for at least one half hour prior to procuring, i.e., harvesting, the organ or organs.

In certain embodiments, the pores have an average pore size of from about 60 kDa to about 150 kDa. In other embodiments, the average pore size is greater than 65 kDa. In yet other embodiments, the average pore size is no greater than 65 kDa. In still further embodiments, the average pore size is from about 60 kDa to about 65 kDa.

In certain embodiments, the donor is a brain-dead donor. In certain embodiments, the donor is a human, e.g., an adult human or a pediatric human. In certain embodiments, the organ or organs is one or more of a heart, lungs, kidney, liver, intestine, or pancreas.

In certain embodiments, the hollow fibers comprise a polymer. In certain embodiments, the polymer is polysulfone. In certain embodiments, the lumen of the hollow fibers has a diameter of from about 100 μM to about 700 μM. In certain embodiments, the diameter is from about 175 μM to about 225 μM. In another embodiment, the diameter is from about 600 μM to about 700 μM. In certain embodiments, the surface area of the hollow fibers is from about 0.01 m² to about 4.0 m², e.g., from about 1.9 m² to about 2.1 m², from about 0.05 m² to about 0.1 m², from about 0.25 m² to about 0.75 m², or from about 1.0 m² to 1.5 m².

In certain embodiments, the cartridge comprises a fluid inlet port and a fluid outlet port and/or the cartridge comprises one or more ports.

In certain embodiments, the proinflammatory molecules removed from the blood are selected from one or more of IL-6, IL-8, TNF-α, IL-1β, MCP-1, CCL2, IP-10 , CXCL10, C3a, C5a, soluble TNF receptor II, matrix metalloproteinase-9, IL-10, soluble gp130, or procalcitonin. In some embodiments, the pro-inflammatory molecule is a microbe, a cell-wall component of a microbe, or lipopolysaccharide.

In certain embodiments, the flow rate of blood through the cartridge is from about 100 mL/min to about 600 mL/min. For example, in some embodiments, the flow rate is from about 100 mL/min to about 400 mL/min, from about 150 mL/min to about 300 mL/min, or from about 150 mL/min to about 250 mL/min.

In certain embodiments, the donor's blood is passed through the cartridge or contacted with the membrane for from about 0.5 to about 120 hours prior to procuring one or more organs from the donor. For example, in some embodiments, the donor's blood is passed through the cartridge or contacted with the membrane for from about 1 hour to about 72 hours. In some embodiments, the donor's blood is passed through the cartridge or contacted with the membrane for from about 1 hour to about 24 hours. In other embodiments, the donor's blood is passed through the cartridge or contacted with the membrane for about 1 hour, from about 3 hours to about 6 hours, from about 6 hours to about 12 hours, about 24 hours, about 48 hours or about 72 hours prior to procuring one or more organs from the donor.

In certain embodiments, after the donor's blood is passed through the cartridge or contacted with the membrane, the donor shows an improvement in one or more of mean arterial pressure, central venous pressure, ejection fraction, arterial blood gas, partial pressure of arterial oxygen to fraction of inspired oxygen ratio (PaO_{2:}FiO₂), serum sodium, urine output, glucose level, hemoglobin level, or reduction in dose or number of vasopressors needed to maintain blood pressure.

In certain embodiments, the cartridge or membrane is connected to the donor via an extracorporeal circuit comprising a line from an artery of the donor, a line to a vein of the donor, and an ultrafiltrate collection container. In certain embodiments, the cartridge or membrane is connected to the donor via an extracorporeal circuit comprising a line from a vein of the donor, a line to a vein of the donor, and an ultrafiltrate collection container. In certain embodiments, the extracorporeal circulation system comprises a double lumen catheter inserted in to a vein of the donor enabling pumping of blood from the vein and returning of blood to the vein. In certain embodiments, the extracorporeal circulation system further comprises one or more of an ultrafiltrate pump, ultrafiltrate pressure sensor, blood sensor, filter pressure sensor, venous pressure sensor, access pressure sensor, IV fluid return pump, or a temperature regulator.

In certain embodiments, the ultrafiltration rate of the cartridge is from about 1 mL/min to about 180 mL/min, e.g., from about 40 mL/min to about 180 mL/min.

In certain embodiments, the method maintains or revitalizes one or more organs of the donor so that the one or more organs is suitable for transplantation to a recipient.

In certain embodiments, the method further comprises the step of procuring one or more organs from the donor.

These and other aspects and features of the invention are described in the following detailed description and claims.

### DESCRIPTION OF THE DRAWINGS

The invention can be more completely understood with reference to the following drawings, in which

FIG. 1 is a schematic representation of an exemplary cartridge;

FIG. 2 is a schematic representation of an exemplary extracorporeal blood circuit used in preparing one or more organs from a donor for transplantation to a recipient;

FIG. 3 is a schematic representation of an exemplary extracorporeal blood circuit used in preparing one or more organs from a donor for transplantation to a recipient;

FIG. 4 is a schematic representation of an exemplary extracorporeal blood circuit used in preparing one or more organs from a donor for transplantation to a recipient; and

FIG. 5 is line graph showing dose of the vasopressor norepinephrine over time for brain-dead organ donor subjects who were subject to the systems and methods described herein.

### DETAILED DESCRIPTION

The invention is based, in part, upon the discovery that organ recovery and transplant outcomes, e.g., the number organs recovered and/or transplanted per donor and the quality of those organs, can be improved by passing blood from a donor, e.g., a brain-dead donor, through a semi-permeable membrane, for example, one or more membranes disposed in a hemofilter. Without wishing to be bound by theory, it is contemplated that processing the donor blood using this approach removes proinflammatory cytokines or other proinflammatory molecules from the blood, including microbes or cell-wall fragments or products of microbes (such as toxins) entering the blood stream from the gut, that negatively impact organ function, recovery and transplantation.

Methods of the invention improve both the quality of organs procured, i.e., harvested, from donors as well as the quantity of organs that can be procured. Organs deemed suitable for donation to a recipient are procured by surgical removal from the donor. According to the data presented herein, on average one additional organ is procured from donors treated according to the methods of the invention as compared to the historic average of approximately 3.8 organs per donor in the United States, and on average 0.5 additional organs are transplanted from donors treated according to the methods of the invention as compared to the historic average of approximately 3.3 organs per donor in the United States. Improving the quality of procured organs also has the beneficial effect of reducing the number of organs that are procured but not ultimately transplanted into recipients and of reducing the number of transplanted organs that are rejected by donors after transplant.

Accordingly, in one aspect, the invention provides a method of preparing one or more organs from a donor for transplantation to a recipient.

In one embodiment, the methods of the invention involve passing blood from a donor through an extracorporeal membrane. The membrane includes a plurality of pores that permit proinflammatory molecules to be removed from the blood so that blood depleted of proinflammatory molecules can be returned to the donor.

In another embodiment, the methods of the invention involve passing blood from the donor through an extracorporeal cartridge comprising a housing and a plurality of semi-permeable hollow fibers disposed therein. Each of the semi-permeable hollow fibers defines a lumen and a plurality of pores, for example, in the wall of the hollow fiber, such that when the blood passes through the lumens of the hollow fibers, proinflammatory molecules from the blood pass through the pores and are removed from the blood. Blood depleted of proinflammatory molecules is returned to the donor. In some embodiments, blood from the donor is passed through the cartridge for at least one half hour prior to procuring the organ or organs.

Various features and aspects of the invention are discussed in more detail below.

I. Membranes/Cartridges

The methods of the invention relate to passing blood from a donor through an extracorporeal membrane. The membrane includes a plurality of pores having an average pore size of at least 60 kDa that, for example, permit proinflammatory molecules to be removed from the blood so that blood depleted of proinflammatory molecules can be returned to the donor.

In certain embodiments, the extracorporeal membrane is disposed in a cartridge. Although the underlying principles for designing an appropriate cartridge are discussed in detail, it is understood that cartridges useful in the practice of the invention are not limited to the particular design configurations discussed herein. In certain embodiments, a cartridge useful in the practice of the invention may, for example, comprise a housing and a plurality of semi-permeable hollow fibers disposed therein, each of the semi-permeable hollow fibers comprising a lumen and a plurality of pores. The cartridge may further comprise a fluid inlet port and a fluid outlet port and/or one or more ultrafiltrate ports.

Other cartridges useful in the practice of the invention include one or more fluid permeable membranes capable of filtering proinflammatory molecules from the blood.

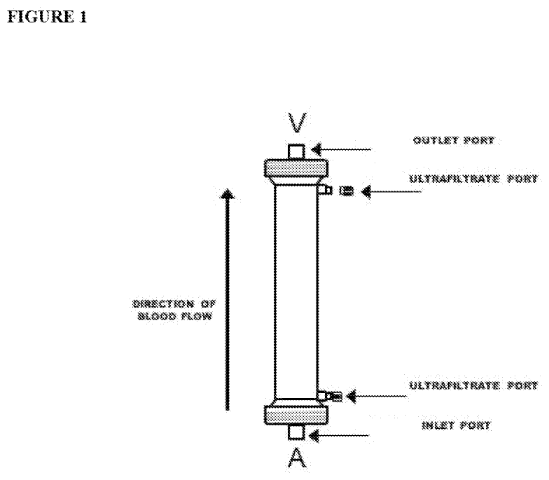

For example, as shown in FIG. 1, blood may enter the cartridge through a fluid inlet port (e.g., an arterial inlet port), pass through the hollow fibers and exit at the opposite end through a fluid outlet port (e.g., a venous outlet port).

It is understood that the membrane or hollow fibers in the cartridge used for filtration are not limited to a particular type, kind or size, and may be made of any appropriate material; however, the material should be biocompatible. For example, a surface of the membrane or fibers may be any biocompatible polymer comprising one or more of nylon, polyethylene, polyurethane, polyethylene terephthalate (PET), polytetrafluoroethylene (PTFE), CUPROPHAN (a cellulose regenerated by means of the cuprammonium process, available from Enka), HEMOPHAN (a modified CUPROPHAN with improved biocompatibility, available from Enka), CUPRAMMONIUM RAYON (a variety of CUPROPHAN, available from Asahi), BIOMEMBRANE (cuprammonium rayon available from Asahi), saponified cellulose acetate (such as fibers available from Teijin or CD Medical), cellulose acetate (such as fibers available from Toyobo Nipro), cellulose (such as that are regenerated by the modified cupramonium process or by means of the viscose process, available from Terumo or Textikombinat (Pima, GDR) respectively), polyacrylonitrile (PAN), polysulfone, polyethersulfone, polyarylethersulfone, acrylic copolymers (such as acrylonitrile-NA-methallyl-sulfonate copolymer, available from Hospal), polycarbonate copolymer (such as GAMBRONE, a fiber available from Gambro), polymethylmethacrylate copolymers (such as fibers available from Toray), ethylene vinyl copolymer (such as EVAL, a ethylene-vinyl alcohol copolymer available from Kuraray), polyvinylalcohol, polyamide, and polycarbonate. Alternatively, a surface may be nylon mesh, cotton mesh, or woven fiber. The surface can have a constant thickness or an irregular thickness. In some embodiments, fibers may include silicon, for example, silicon nanofabricated membranes (see, e.g., U.S. Patent Publication No. 2004/0124147). In certain embodiments, the membrane or hollow fibers in the cartridge used for filtration include a polysulfone, e.g., glycerin-free polysulfone. Other suitable biocompatible fibers are known in the art, for example, in Salem and Mujais (1993) DIALYSIS THERAPY 2D ED., Ch. 5: Dialyzers, Eds. Nissensen and Fine, Hanley & Belfus, Inc., Philadelphia, Pa.

Depending upon the donor and the application, the surface area of the membrane or hollow fibers in the cartridge used for filtration may be from about 0.01 m² to about 4.0 m². For example, the surface area of the hollow fibers may be from about 0.01 m² to about 3.0 m², about 0.01 m² to about 2.0 m², about 0.01 m² to about 1.0 m², about 0.01 m² to about 0.5 m², about 0.01 m² to about 0.1 m², about 0.01 m² to about 0.05 m², about 0.05 m² to about 4.0 m², about 0.05 m² to about 3.0 m², about 0.05 m² to about 2.0 m², about 0.05 m² to about 1.0 m², about 0.05 m² to about 0.5 m², about 0.05 m² to about 0.1 m², about 0.1 m² to about 4.0 m², about 0.1 m² to about 3.0 m², about 0.1 m² to about 2.0 m², about 0.1 m² to about 1.0 m², about 0.1 m² to about 0.5 m², about 0.2 m² to about 0.4 m², about 0.25 m² to about 0.35 m², about 0.5 m² to about 4.0 m², about 0.5 m² to about 3.0 m², about 0.5 m² to about 2.0 m², about 0.5 m² to about 1.0 m², about 0.5 m² to about 0.75 m², about 1.0 m² to about 4.0 m², about 1.0 m² to about 3.0 m², about 1.0 m² to about 2.0 m², about 1.0 m² to about 1.5 m², about 1.75 m² to about 2.5 m², about 1.75 m² to about 2.25 m², about 2.0 m² to about 4.0 m², about 2.0 m² to about 3.0 m², or about 3.0 m² to about 4.0 m². In certain embodiments, the surface area of the hollow fibers is from about 1.9 m² to about 2.1 m², from about 0.05 m²to about 0.1 m², from about 0.25 m² to about 0.75 m², or from about 1.0 m²to 1.5 m². In certain embodiments, the surface area is about 2.0 m². It will be appreciated that the surface area will vary depending on the age and size of the donor. For example, pediatric and infant donors will require cartridges with smaller surface areas as compared to adult donors; smaller adults may also require cartridges with smaller surface areas as compared to larger adults.

The surface area of the membrane or hollow fibers can be adapted by lengthening or shortening the length of the membrane or fibers. For example, the length of the membrane or hollow fibers may be about 30 cm, about 29 cm, about 28 cm, about 27 cm, about 26 cm, about 25 cm, about 24 cm, about 23 cm, about 22 cm, about 21 cm, about 20 cm, about 19 cm, about 18 cm, about 17 cm, about 16 cm, about 15 cm, about 14 cm, about 13 cm, about 12 cm, about 11 cm, or about 10 cm.

The surface area of the hollow fibers can also be adapted by varying the number of hollow fibers used in the cartridge. In certain embodiments, the cartridge comprises from about 9,000 to about 15,000 hollow fibers. For example, the cartridge may comprise from about 9,000 to about 14,000, from about 9,000 to about 13,000, from about 9,000 to about 12,000, from about 9,000 to about 11,000, from about 9,000 to about 10,000, from about 10,000 to about 15,000, from about 10,000 to about 14,000, from about 10,000 to about 13,000, from about 10,000 to about 12,000, from about 10,000 to about 11,000, from about 11,000 to about 15,000, from about 11,000 to about 14,000, from about 11,000 to about 13,000, from about 11,000 to about 12,000, from about 12,000 to about 15,000, from about 12,000 to about 14,000, from about 12,000 to about 13,000, from about 12,000 to about 15,000, from about 12,000 to about 14,000, or from about 14,000 to about 13,000 hollow fibers.

Also depending upon the donor and the application, the lumen of hollow fibers in the cartridge may be from about 100 μM to about 700 μM. For example, the lumen may be from about 100 μM to about 700 μM, about 100 μM to about 600 μM, about 100 μM to about 500 μM, about 100 μM to about 400 μM, about 100 μM to about 300 μM, about 100 μM to about 200 μM, about 200 μM to about 700 μM, about 200 μM to about 600 μM, about 200 μM to about 500 μM, about 200 μM to about 400 μM, about 200 μM to about 300 μM, about 300 μM to about 700 μM, about 300 μM to about 600 μM, about 300 μM to about 500 μM, about 300 μM to about 400 μM, about 400 μM to about 700 μM, about 400 μM to about 600 μM, about 400 μM to about 500 μM, about 500 μM to about 700 μM, about 500 μM to about 600 μM, or about 600 μM to about 700 μM. In certain embodiments, the lumen of the hollow fibers has a diameter of about 175 μM to about 225 μM, or about 600 μM to about 700 μM. In certain embodiments, the lumen of the hollow fibers has a diameter of about 200 μM.

In certain embodiments, the hollow fibers are made of a semi-permeable membrane. The term “membrane” refers to a surface capable of receiving a fluid on both sides of the surface, or a fluid on one side and gas on the other side of the surface. It is understood that the sieving characteristics of a membrane depend not only on the pore size, but also on the physical, chemical, and electrical characteristics of the material from which the fiber or membrane is made, the particular manufacturing technique used and post production processing (e.g. sterilization). Nonetheless, the size of a pore in a porous membrane or fiber can be represented by a molecular weight cutoff (MWC), i.e., the lowest molecular weight of solute in which 90% of the solute is retained by the membrane or fiber. Molecular weight cutoff may be measured by any method known in the art, including, for example, exposing the membrane or fiber to a solute with a known molecular weight (e.g., a polyethylene glycol or dextran) and ascertaining retention of the solute by the membrane or fiber. It is understood that the molecular weight cutoff may vary depending upon the conditions in which it is measured, for example, the molecular weight cutoff of a membrane or fiber that is measured when the membrane or fiber is disposed in an extracorporeal circuit including subject blood (i.e. the effective molecular weight cutoff) may be lower than the molecular weight cutoff of the membrane or fiber that is measured in a test situation (i.e. the nominal effective molecular weight cutoff).

A membrane or fiber can be porous (e.g., selectively porous or semi-porous) such that it is capable of fluid or gas flow therethrough. It is understood that the term “porous” as used herein to describe a surface, fiber, or membrane includes generally porous, selectively porous and/or semi-porous surfaces or membranes. A semi-permeable membrane refers to a membrane that permits only certain molecules to pass through while being impermeable to other molecules.

In one embodiment, a membrane is semi-permeable based on the size of molecules contacting the membrane. For example, in one embodiment, a semi-permeable membrane is permeable to molecules below a certain size threshold while molecules above that size threshold are excluded from passing through the membrane.

Accordingly, in certain embodiments, the semi-permeable membrane or hollow fibers in the cartridge used for filtration of donor blood comprise a plurality of pores with an average pore size of from about 60 kDa to about 150 kDa. For example, the plurality of pores may have an average pore size of about 65 kDa to about 150 kDa, about 70 kDa to about 150 kDa, about 80 kDa to about 150 kDa, about 90 kDa to about 150 kDa, about 100 kDa to about 150 kDa, about 110 kDa to about 150 kDa, about 120 kDa to about 150 kDa, about 130 kDa to about 150 kDa, about 140 kDa to about 150 kDa, about 60 kDa to about 140 kDa, about 65 kDa to about 140 kDa, about 70 kDa to about 140 kDa, about 80 kDa to about 140 kDa, about 90 kDa to about 140 kDa, about 100 kDa to about 140 kDa, about 110 kDa to about 140 kDa, about 120 kDa to about 140 kDa, about 130 kDa to about 140 kDa, about 60 kDa to about 130 kDa, about 65 kDa to about 130 kDa, about 70 kDa to about 130 kDa, about 80 kDa to about 130 kDa, about 90 kDa to about 130 kDa, about 100 kDa to about 130 kDa, about 110 kDa to about 130 kDa, about 120 kDa to about 130 kDa, about 60 kDa to about 120 kDa, about 65 kDa to about 120 kDa, about 70 kDa to about 120 kDa, about 80 kDa to about 120 kDa, about 90 kDa to about 120 kDa, about 100 kDa to about 120 kDa, about 110 kDa to about 120 kDa, about 60 kDa to about 110 kDa, about 65 kDa to about 110 kDa, about 70 kDa to about 110 kDa, about 80 kDa to about 110 kDa, about 90 kDa to about 110 kDa, about 100 kDa to about 110 kDa, about 60 kDa to about 100 kDa, about 65 kDa to about 100 kDa, about 70 kDa to about 100 kDa, about 80 kDa to about 100 kDa, about 90 kDa to about 100 kDa, about 60 kDa to about 90 kDa, about 65 kDa to about 90 kDa, about 70 kDa to about 90 kDa, about 80 kDa to about 90 kDa, about 60 kDa to about 80 kDa, about 65 kDa to about 80 kDa, about 70 kDa to about 80 kDa, about 60 kDa to about 70 kDa, about 65 kDa to about 70 kDa, or about 60 kDa to about 65 kDa. The plurality of pores may have an average pore size greater than 60 kDa. The plurality of pores may have an average pore size greater than 65 kDa. The plurality of pores may have an average pore size greater than 70 kDa. The plurality of pores may have an average pore size greater than 80 kDa, greater than 90 kDa, greater than 100 kDa, greater than 110 kDa, greater than 120 kDa, greater than 130 kDa, greater than 140 kDa, or greater than 150 kDa. The plurality of pores may have an average pore size no greater than 65 kDa. The plurality of pores may have an average pore size from about 60 kDa to about 65 kDa.

As used herein, the sieving coefficient (SC) of a membrane or fiber for a given solute refers to the ratio between the solute concentration in the filtrate and its concentration in the feed (e.g., blood, plasma, or plasma water). An SC of 1 indicates unrestricted transport while an SC of 0 indicates no transport at all. SC is specific for each fiber or membrane for each solute. It is understood that SC varies depending upon the treatment conditions, and measurement of the SC may even vary during treatment because the characteristics of the fiber or membrane may change.

In certain embodiments, the membrane or hollow fibers in the cartridge used for filtration have a sieving coefficient for IL-6 of about 0.8 to about 1.5. For example, the sieving coefficient for IL-6 may be from about 0.8 to about 1.4, about 0.8 to about 1.3, about 0.8 to about 1.2, about 0.8 to about 1.1, about 0.8 to about 1.0, about 0.8 to about 0.9, about 0.9 to about 1.5, about 0.9 to about 1.4, about 0.9 to about 1.3, about 0.9 to about 1.2, about 0.9 to about 1.1, about 0.9 to about 1.0, about 1.0 to about 1.5, about 1.0 to about 1.4, about 1.0 to about 1.3, about 1.0 to about 1.2, about 1.0 to about 1.1, about 1.1 to about 1.5, about 1.1 to about 1.4, about 1.1 to about 1.3, about 1.1 to about 1.2, about 1.2 to about 1.5, about 1.2 to about 1.4, about 1.2 to about 1.3, about 1.3 to about 1.5, about 1.3 to about 1.4, or about 1.4 to about 1.5. In certain embodiments, the sieving coefficient for IL-6 is at least 0.8, 0.9, 1.0, 1.1, 1.2, 1.3, 1.4, or 1.5. In certain embodiments, the sieving coefficient for IL-6 is about 1.25. The sieving coefficient for IL-6 may be measured as described in Clar et al. (1997) ASAIO J 43:163-170.

In certain embodiments, the membrane or hollow fibers in the cartridge used for filtration have a sieving coefficient for urea of about 0.8 to about 1.0. For example, the sieving coefficient for urea may be from about 0.8 to about 1.0, from about 0.8 to about 0.9, or from about 0.9 to about 1.0. In certain embodiments, the sieving coefficient for urea is at least 0.8, 0.9, or 1.0. In certain embodiments, the sieving coefficient for urea is about 1.0. The sieving coefficient for urea may be measured in aqueous solution at a flow rate of 200 mL/min and transmembrane pressure (TMP) of 50 mmHg.

In certain embodiments, the membrane or hollow fibers in the cartridge used for filtration have a sieving coefficient for creatinine of about 0.8 to about 1.0. For example, the sieving coefficient for creatinine may be from about 0.8 to about 1.0, from about 0.8 to about 0.9, or from about 0.9 to about 1.0. In certain embodiments, the sieving coefficient for creatinine is at least 0.8, 0.9, or 1.0. In certain embodiments, the sieving coefficient for creatinine is about 1.0. The sieving coefficient for creatinine may be measured in aqueous solution at a flow rate of 200 mL/min and transmembrane pressure (TMP) of 50 mmHg.

In certain embodiments, the membrane or hollow fibers in the cartridge used for filtration have a sieving coefficient for vitamin B12 of about 0.8 to about 1.0. For example, the sieving coefficient for vitamin B12 may be from about 0.8 to about 1.0, from about 0.8 to about 0.9, or from about 0.9 to about 1.0. In certain embodiments, the sieving coefficient for creatinine vitamin B12 is at least 0.8, 0.9, or 1.0. In certain embodiments, the sieving coefficient for vitamin B12 is about 1.0. The sieving coefficient for vitamin B12 may be measured in aqueous solution at a flow rate of 200 mL/min and transmembrane pressure (TMP) of 50 mmHg.

In certain embodiments, the membrane or hollow fibers in the cartridge used for filtration have a sieving coefficient for myoglobin of about 0.10 to about 0.25. For example, the sieving coefficient for myoglobin may be from about 0.10 to about 0.25, about 0.10 to about 0.20, about 0.10 to about 0.15, about 0.15 to about 0.25, about 0.15 to about 0.20, or about 0.20 to about 0.25. In certain embodiments, the sieving coefficient for myoglobin is at least 0.10, 0.15, 0.20, or 0.25. In certain embodiments, the sieving coefficient for myoglobin is about 0.17.

The sieving coefficient for myoglobin may be measured in bovine blood at a flow rate of 400 mL/min and transmembrane pressure (TMP) of 400 mmHg.

In certain embodiments, the membrane or hollow fibers in the cartridge used for filtration have a sieving coefficient for albumin of about 0.005 to about 0.025. For example, the sieving coefficient for albumin may be from about 0.005 to about 0.025, about 0.005 to about 0.020, about 0.005 to about 0.015, about 0.005 to about 0.010, about 0.010 to about 0.025, about 0.010 to about 0.020, about 0.010 to about 0.015, about 0.015 to about 0.025, about 0.015 to about 0.020, or about 0.020 to about 0.025. In certain embodiments, the sieving coefficient for albumin is at least 0.005, 0.010, 0.015, 0.020, or 0.025. In certain embodiments, the sieving coefficient for albumin is about 0.015. In certain embodiments, the membrane or hollow fibers in the cartridge used for filtration have a sieving coefficient for albumin of about 0.2 to about 1.0. For example, the sieving coefficient for albumin may be from about 0.2 to about 1.0, about 0.2 to about 0.8, about 0.2 to about 0.6, about 0.2 to about 0.4, about 0.4 to about 1.0, about 0.4 to about 0.8, about 0.4 to about 0.6, about 0.6 to about 1.0, about 0.6 to about 0.8, or about 0.8 to about 1.0. The sieving coefficient for albumin may be measured in bovine blood at a flow rate of 400 mL/min and transmembrane pressure (TMP) of 400 mmHg.

The housing of the cartridge is not limited to a particular set of dimensions (e.g., length, width, weight, or another dimension). It is understood that the size and shape of the housing of the cartridge may be designed to provide the appropriate fill volume and to minimize turbulence when a fluid is passed through the cartridge. Furthermore, it is understood that the size, shape and composition of the membrane located within the cartridge may be designed to provide the appropriate surface area and to minimize turbulence when a fluid is passed through the cartridge. Also, the size of the cartridge depends upon the size of the donor. For example, pediatric and infant donors will smaller cartridges areas as compared to adult donors; smaller adults may also require smaller cartridges as compared to larger adults.

The housing of the cartridge can be fabricated from a variety of materials, but the material that defines that fluid contacting surface in the inner volume should be biocompatible. The cartridge can be constructed from a variety of materials including, metals such as titanium, or stainless steel with or without surface coatings of refractory metals including titanium, tantalum, or niobium; ceramics such as alumina, silica, or zirconia; polymers, such as polyvinylchloride, polyethylene, or polycarbonate; or plastic.

It is understood that the cartridge, once fabricated, should be sterilized prior to use. Sterility can be achieved through exposure to one or more sterilizing agents, separately or in combination, such as high temperature, high pressure, radiation, or chemical agents such as ethylene oxide, for example. The cartridge preferably is sterilized once it has been packaged, for example, after it has been hermetically sealed within an appropriate container (i.e., the cartridge is terminally sterilized). The sterilization process preferably achieves a sterility assurance level (SAL) of 10⁻³ or less; i.e., the probability of any given unit being nonsterile after the process is no more than 1 in 10³. More preferably, the sterilization process achieves an SAL of no more than 10⁻⁴, no more than 10⁻⁵, or no more than 10⁻⁶.

In certain embodiments, the cartridge is the CLR 2.0 filter (SeaStar Medical, Inc., Cardiff-by the-Sea, Calif.).

II. Blood Circuits

It is understood that a membrane or cartridge can be used in a variety of different fluid circuits or extracorporeal circulation system depending upon the intended use. The fluid circuits generally are configured to prepare one or more organs from a donor for transplantation to a recipient by hemofiltration.

In basic form, an exemplary circuit includes a cartridge, a fluid connection for blood to flow from a blood source (for example, an artery or vein in a subject, such as an organ donor) to the cartridge, and a fluid connection for treated blood to flow from the cartridge to a receptacle (for example, back to a vein in the organ donor).

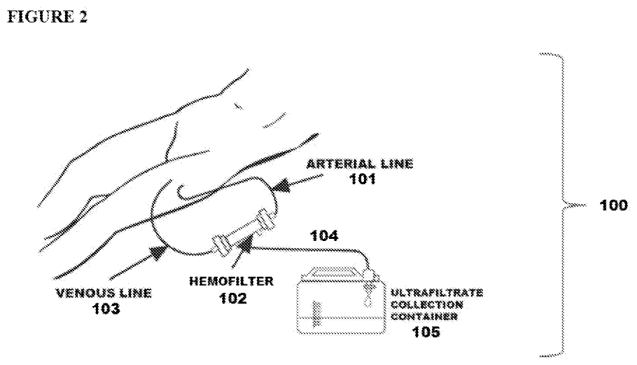

For example, as shown in the exemplary circuit 100 depicted in FIG. 2, blood flows from an artery in a donor to a blood line 101. Blood then enters the cartridge 102, passes through the hollow fibers (which run parallel to the length of the cartridge), and exits at the opposite end. Blood then flows through a blood line 103 and is returned to a vein in the donor. Hemofiltration occurs while the blood passes through the cartridge 102, with ultrafiltrate leaving the cartridge and passing through a line 104 to an ultrafiltrate collection container 105. During hemofiltration, plasma water and non-protein bound plasma solutes, including proinflammatory molecules, are removed from the blood by ultrafiltration, while cellular elements and larger proteins are returned to the donor. For an exemplary cartridge, ultrafiltration occurs as a result of pressure gradients across the porous hollow fiber membrane. This gradient is achieved by: (1) positive pressure in the blood compartment, provided by the donor's mean arterial blood pressure and (2) negative pressure in the filtrate compartment, provided by the modest siphoning effect generated when an ultrafiltrate collection container is placed below the hemofilter. Replacement or substitution fluid may additionally be infused into the circuit 100 at a controlled rate to maintain fluid, electrolyte, acid base and nitrogen balance.

In certain embodiments, the extracorporeal circulation system further comprises one or more of an ultrafiltrate pump, ultrafiltrate pressure sensor, blood sensor, filter pressure sensor, venous pressure sensor, access pressure sensor, IV fluid return pump, or a temperature regulator.

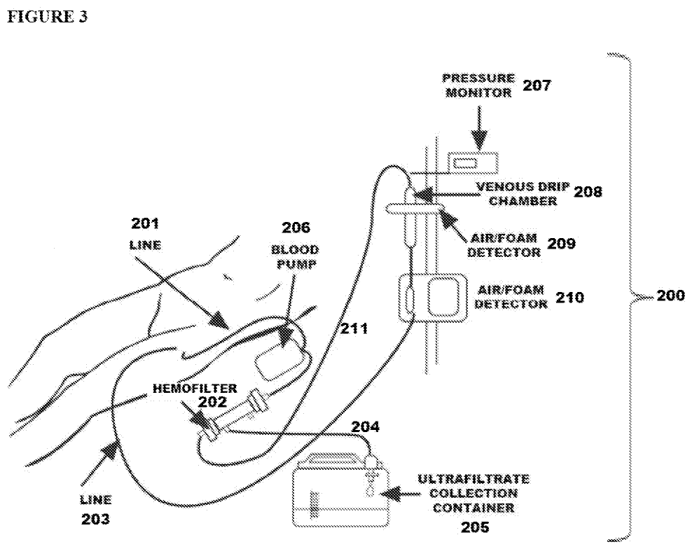

For example, in certain embodiments, a minimum flow rate is required for proper operation of the cartridge, and therefore one or more pumps may be necessary in donors with systolic blood pressures below a certain threshold. For example, a pump assisted circuit 200 is shown in FIG. 3. Blood flows from an artery or vein in a donor to a blood line 201. Blood enters a pump 206 before continuing to the cartridge 202. Flow rates at the pump 206 can be chosen at ranges described herein. Blood enters the cartridge 202, passes through the hollow fibers, and exits at the opposite end. Hemofiltration occurs while the blood passes through the cartridge, with ultrafiltrate leaving the cartridge and passing through a line 204 to an ultrafiltrate collection container 205. Blood then flows through a blood line 211 and, prior to returning to a vein in the donor, passes through a venous drip chamber 208. The system may also include a pressure monitor 209 and air/foam detector 220.

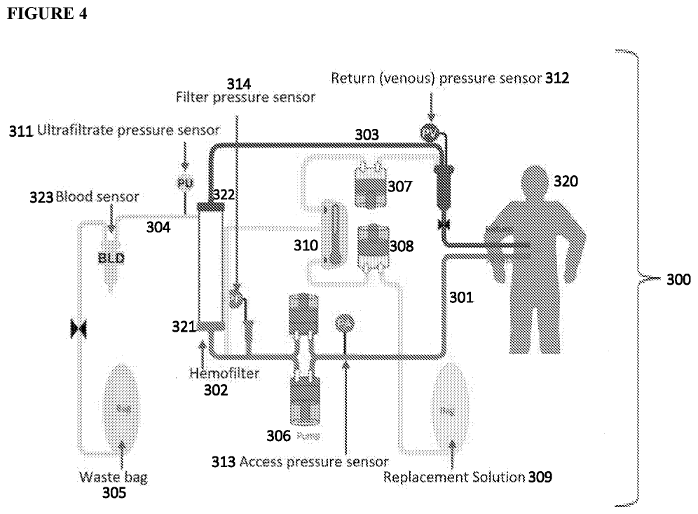

An additional exemplary circuit 300 is shown in FIG. 4. Blood flows from a donor 300 to a blood line 301. Blood enters a pump 306 before continuing to the cartridge 302. Flow rates at the pump 306 can be chosen at ranges described herein. Pressure is sampled at an access pressure sensor 313 prior to the pump, and a filter pressure sensor 314 after the pump and prior to the cartridge 302. Blood enters the cartridge 302 via a fluid inlet port 321, passes through the hollow fibers, and exits at the opposite end via a fluid outlet port 322. Hemofiltration occurs while the blood passes through the cartridge 302, with ultrafiltrate leaving the cartridge and passing through an ultrafiltrate line 304 to a waste bag 305. The ultrafiltrate line 304 may include an ultrafiltrate pressure sensor 311 and/or a blood sensor 323. Filtered blood leaves the cartridge 302 and flows through a blood line 303 to the donor. Pressure may be sampled at a return pressure sensor 312 prior to the returning to the donor. The system may also include a source of replacement solution. Replacement solution flows from a bag 309 to a pump 308, and is introduced into the circuit immediately prior to the cartridge and/or immediately prior to the return of fluid to the donor. Replacement solution temperature is monitored by a temperature regulator 310.

A cartridge may be connected to the donor's vascular system via vascular access which may include: arteriovenous femoral catheters, arteriovenous jugular catheters, Quinton-Scribner Shunt, arteriovenous fistula, veno-venous femoral catheters, veno-venous jugular catheters, veno-venous subclavian catheters, or veno-venous catheters at other sites. In certain embodiments, this is accomplished with a percutaneous catheter arrangement or an arteriovenous shunt. In certain embodiments, the extracorporeal circulation system comprises a double lumen catheter inserted in to a vein enabling pumping of blood from the vein and returning of blood to the vein.

The rate of blood flowing through the system will depend on the condition of the donor, the molecular weight cutoff of the associated fibers, the body size of donor, and other requirements for effective preparation of organs for transplant. The amount of blood, the blood flow rate and the duration of treatment are preferably determined on a case by case basis after factoring the weight, the age and the nature of the donor.

In certain embodiments, the blood flow rate through the cartridge is from about 100 mL/min to about 600 mL/min. For example the blood flow rate may be from about 200 mL/min to about 600 mL/min, about 300 mL/min to about 600 mL/min, about 400 mL/min to about 600 mL/min, about 500 mL/min to about 600 mL/min, about 100 mL/min to about 500 mL/min, about 200 mL/min to about 500 mL/min, about 300 mL/min to about 500 mL/min, about 400 mL/min to about 500 mL/min, about 100 mL/min to about 400 mL/min, about 200 mL/min to about 400 mL/min, about 300 mL/min to about 400 mL/min, about 100 mL/min to about 300 mL/min, about 200 mL/min to about 300 mL/min, or about 100 mL/min to about 200 mL/min. In certain embodiments, the blood flow rate is from about 100 mL/min to about 400 mL/min. In certain embodiments, the blood flow rate is from about 150 mL/min to about 250 mL/min. In certain embodiments, the blood flow rate is from about 135 mL/min to about 150 mL/min.

In certain embodiments, the ultrafiltration rate of the cartridge is from about 0 mL/min to about 180 mL/min, e.g., about 1 mL/min to about 180 mL/min. For example the ultrafiltration rate may be from about 1 mL/min to about 180 mL/min, about 5 mL/min to about 180 mL/min, about 20 mL/min to about 180 mL/min, about 40 mL/min to about 180 mL/min, about 60 mL/min to about 180 mL/min, about 80 mL/min to about 180 mL/min, about 100 mL/min to about 180 mL/min, about 120 mL/min to about 180 mL/min, about 140 mL/min to about 180 mL/min, about 160 mL/min to about 180 mL/min, about 1 mL/min to about 160 mL/min, about 5 mL/min to about 160 mL/min, about 20 mL/min to about 160 mL/min, about 40 mL/min to about 160 mL/min, about 60 mL/min to about 160 mL/min, about 80 mL/min to about 160 mL/min, about 100 mL/min to about 160 mL/min, about 120 mL/min to about 160 mL/min, about 140 mL/min to about 160 mL/min, about 1 mL/min to about 140 mL/min, about 5 mL/min to about 140 mL/min, about 20 mL/min to about 140 mL/min, about 40 mL/min to about 140 mL/min, about 60 mL/min to about 140 mL/min, about 80 mL/min to about 140 mL/min, about 100 mL/min to about 140 mL/min, about 120 mL/min to about 140 mL/min, about 1 mL/min to about 120 mL/min, about 5 mL/min to about 120 mL/min, about 20 mL/min to about 120 mL/min, about 40 mL/min to about 120 mL/min, about 60 mL/min to about 120 mL/min, about 80 mL/min to about 120 mL/min, about 100 mL/min to about 120 mL/min, about 1 mL/min to about 100 mL/min, about 5 mL/min to about 100 mL/min, about 20 mL/min to about 100 mL/min, about 40 mL/min to about 100 mL/min, about 60 mL/min to about 100 mL/min, about 80 mL/min to about 100 mL/min, about 1 mL/min to about 80 mL/min, about 5 mL/min to about 80 mL/min, about 20 mL/min to about 80 mL/min, about 40 mL/min to about 80 mL/min, about 60 mL/min to about 80 mL/min, about 1 mL/min to about 60 mL/min, about 5 mL/min to about 60 mL/min, about 20 mL/min to about 60 mL/min, about 40 mL/min to about 60 mL/min, about 1 mL/min to about 40 mL/min, about 5 mL/min to about 40 mL/min, about 20 mL/min to about 40 mL/min, about 1 mL/min to about 20 mL/min, about 5 mL/min to about 20 mL/min, or about 1 mL/min to about 5 mL/min. In certain embodiments, the ultrafiltration rate of the cartridge is from about 40 mL/min to about 180 mL/min.

In certain embodiments, the donor's blood is passed through the cartridge or contacted with the membrane for from about 0.5 to about 120 hours prior to procuring one or more organs from the donor. For example, the donor's blood may be passed through the cartridge from about 0.5 to about 120, from about 0.5 to about 96, from about 0.5 to about 72, from about 0.5 to about 48, from about 0.5 to about 24, from about 0.5 to about 12, from about 0.5 to about 6, from about 0.5 to about 3, from about 0.5 to about 1, from about 1 to about 120, from about 1 to about 96, from about 1 to about 72, from about 1 to about 48, from about 1 to about 24, from about 1 to about 12, from about 1 to about 6, from about 1 to about 3, from about 3 to about 120, from about 3 to about 96, from about 3 to about 72, from about 3 to about 48, from about 3 to about 24, from about 3 to about 12, from about 3 to about 6, from about 6 to about 120, from about 6 to about 96, from about 6 to about 72, from about 6 to about 48, from about 6 to about 24, from about 6 to about 12, from about 12 to about 120, from about 12 to about 96, from about 12 to about 72, from about 12 to about 48, from about 12 to about 24, from about 24 to about 120, from about 24 to about 96, from about 24 to about 72, from about 24 to about 48, from about 48 to about 120, from about 48 to about 96, from about 48 to about 72, from about 72 to about 120, from about 72 to about 96, from about 96 to about 120 hours. In certain embodiments, the donor's blood is passed through the cartridge or contacted with the membrane for about 1 to about 72 hours, about 1 to about 24 hours, about 1 hour, about 3 to about 6 hours, about 6 to about 12 hours, about 24 hours, or about 48 hours or about 72 hours. In one embodiment, the donor's blood is contact with the membrane or passed through the cartridge from about 6 to about 12 hours. In one embodiment, the donor's blood is contact with the membrane or passed through the cartridge from about 3 to about 6 hours. In one embodiment, the donor's blood is contact with the membrane or passed through the cartridge for about 12 to about 24 hours.

The composition of the material making up the blood pump, ultrafiltrate pump, IV fluid return pump, or tubing is preferably a biocompatible material, for example, polyvinylchloride. The tubing may be flexible and have dimensions complementary with associated hemofilter connections, ultrafiltrate recycling device connections, replacement fluid reservoir connection, joints, stop cocks, or pump heads.

In certain embodiments, fluid circuits incorporating the membrane or cartridge optionally can also perform other blood treatments. For example, fluid circuits optionally can further include additional devices that can filter, oxygenate, warm, or otherwise treat the blood before or after the blood enters the cartridge.

In certain embodiments, the membranes, cartridges and/or the fluid circuits incorporating the membranes or cartridges are controlled by a processor (e.g., computer software). In such embodiments, a device can be configured to detect changes within a donor and provide such information to the processor. In some embodiments, the fluid circuit can automatically process the donor's blood through the cartridge in response to such information. In other embodiments, a health professional is alerted and initiates treatment.

Exemplary membranes, cartridges, and blood circuits that may be useful in the practice of the invention are disclosed, for example, in U.S. Pat. Nos. 8,597,516 and 6,787,040.

III. Therapeutic Uses

The methods disclosed herein can be used to prepare one or more organs from a donor for transplantation to a recipient. In certain embodiments, the organ or organs is one or more of a heart, lungs, kidney, liver, intestine, or pancreas.

As used herein, the terms “subject” and “donor” are used interchangeably and refer to an organism that is an organ donor and is to be treated by the methods and compositions described herein. Such organisms preferably include, but are not limited to, mammals (e.g., murines, primates (e.g., simians), equines, bovines, porcines, canines, felines, and the like), and more preferably includes humans. In a preferred embodiment, the donor is a human donor. In certain embodiments, the donor is a brain-dead donor. Brain-dead donors may include both adult and pediatric donors.

In certain embodiments, the methods disclosed herein increase the likelihood that an organ from the donor will be deemed suitable for donation to a recipient and/or the method revitalizes one or more organs of the donor. Revitalization of organs refers to improving an organ's quality such that it qualifies for procuring for donation whereas prior to treatment according to the methods of invention, the organ would not have qualified for procuring for donation. Whether an organ has been revitalized is generally measured by whether or not a donor meets donor management goals for donation of a specific organ. Donor management goals vary from country to country and within regions of countries, for example, in the United States, but generally set forth a series of critical care end points clinically determined necessary to deem an organ suitable for donation. These endpoints reflect the hemodynamic, respiratory, endocrine, hematologic, acid-base, and renal status of the potential donor.

For example, in certain embodiments, the method results in the donor showing an improvement in one or more of the following critical care end points after their blood has been treated according to the methods of the invention: mean arterial pressure (MAP), central venous pressure (CVP), ejection fraction, arterial blood gas (ABG) pH, partial pressure of arterial oxygen to fraction of inspired oxygen ratio (PaO_{2:}FiO₂), serum sodium, urine output, glucose level, hemoglobin level, or reduction in dose or number of vasopressors needed to maintain blood pressure. For example, in certain embodiments, the method results in the donor having an MAP of 60-100 mmHg. In certain embodiments, the method results in the donor having a CVP of 4-10 mmHg. In certain embodiments, the method results in the donor having an ejection fraction of greater than 50%. In certain embodiments, the method results in the donor having an ABG pH of 7.3-7.45. In certain embodiments, the method results in the donor having a PaO_{2:}FiO₂ of greater than 300 on 5 of positive end-expiratory pressure (PEEP). In certain embodiments, the method results in the donor having serum sodium of 135-155 mEq/L. In certain embodiments, the method results in the donor having blood glucose of less than 150 mg/dl. In certain embodiments, the method results in the donor having a hemoglobin level of less than 10 mg/dl. In certain embodiments, the method results in the donor having urine output greater than 0.5 mL/kg/h for four hours. In certain embodiments, the method results in the donor requiring 1 low dose or less of vasopressor. In one embodiment, the donor meets one or more of the aforementioned critical care endpoints. In one embodiment, the donor meets two or more of the aforementioned critical care endpoints. In one embodiment, the donor meets three or more of the aforementioned critical care endpoints. In one embodiment, the donor meets four or more of the aforementioned critical care endpoints. In another embodiment, the donor meets five or more of the aforementioned critical care endpoints. In yet another embodiment, the donor meets six or more of the aforementioned critical care endpoints. In one embodiment, the donor meets seven or more of the aforementioned critical care endpoints. In one embodiment, the donor meets eight or more of the aforementioned critical care endpoints. In one embodiment, the donor meets nine or more of the aforementioned critical care endpoints. In one embodiment, the donor meets all of the aforementioned critical care endpoints. The more endpoints met by a donor, the greater the likelihood one or more organs will be deemed suitable for procuring for transplant and the greater the likelihood the procured organs will be transplanted into a recipient. In many cases, meeting 8 of the 10 aforementioned critical care endpoints is deemed necessary for procuring of donor organs. Additional measures of suitability for organ donation are described in Malinoski et al. (2012) Crit Care Med. 40(10):2773-80, Malinoski et al. (2011) J Trauma 71(4):990-5, and Patel et al. (2014) JAMA Surg 149(9):969-75.

In certain embodiments, donors treated according to the methods of the invention maintain one or more critical care end points required for organ donation attained prior to treatment with the methods of the invention such as an MAP of 60-100 mmHg, a CVP of 4-10 mmHg, an ejection fraction of greater than 50%, an ABG pH of 7.3-7.45, a PaO_{2:}FiO₂ of greater than 300 on 5 of positive end-expiratory pressure (PEEP), a serum sodium level of 135-155 mEq/L, a blood glucose level of less than 150 mg/dl, a hemoglobin level of less than 10 mg/dl, urine output greater than 0.5 mL/kg/h for four hours, or the need for 1 low dose or less of vasopressor. In other words, treatment of a donor according to methods of the invention allows the donor to maintain one or more critical care end points required for organ donation as opposed to the end point value falling out of the required range if treatment is not administered. In one embodiment, the donor maintains one or more of the aforementioned critical care endpoints. In one embodiment, the donor maintains two or more of the aforementioned critical care endpoints. In one embodiment, the donor maintains three or more of the aforementioned critical care endpoints. In one embodiment, the donor maintains four or more of the aforementioned critical care endpoints. In another embodiment, the donor maintains five or more of the aforementioned critical care endpoints. In yet another embodiment, the donor maintains six or more of the aforementioned critical care endpoints. In one embodiment, the donor maintains seven or more of the aforementioned critical care endpoints. In one embodiment, the donor maintains eight of the aforementioned critical care endpoints. In one embodiment, the donor maintains nine of the aforementioned critical care endpoints. In one embodiment, the donor maintains ten of the aforementioned critical care endpoints. In such embodiments, the suitability of one or more organs for procuring may be maintained by treatment of the donor according to the methods of the invention.

The methods described herein may reduce systemic inflammation in a brain-dead donor. For example, methods and cartridges described herein may reduce a level of a proinflammatory molecule in a donor, e.g., in a body fluid (e.g., blood, plasma, serum, or urine), tissue and/or cell in a donor, e.g., by at least about 10%, about 20%, about 30%, about 40%, about 50%, about 60%, about 70%, about 80%, about 90%, about 95%, or more, relative to levels in an untreated or control subject. For example, methods and cartridges described herein may reduce a level of a pro-inflammatory cytokine or chemokine. Exemplary pro-inflammatory cytokines or chemokines include IL-1-β, IL-4, IL-5, IL-6, IL-8, IL-12, IL-13, IL-17, IL-21, IL-22, IL-23, IL-27, IFN, CCL-2, CCL-3, CCL-5, CCL-20, CXCL-5, CXCL-10, CXCL-12, CXCL-13, and TNF-α. Additional exemplary proinflammatory molecules include MCP-1, IP-10, C3a, C5a, soluble TNF receptor II, matrix metalloproteinase-9, IL-10, soluble gp130, lipopolysaccharide, and procalcitonin. It is understood that reference to a proinflammatory molecule includes the proinflammatory molecule in both an unbound state or in complex with a corresponding ligand. Exemplary proinflammatory molecule-ligand complexes include an IL-6/IL-6 soluble receptor complex, a TNF-a/soluble TNF receptor complex, and an albumin/proinflammatory cytokine complex. In some embodiments, the pro-inflammatory molecule is a microbe, a cell-wall component of a microbe, or endotoxin such as lipopolysaccharide.

The methods described herein can be used alone or in combination with other therapeutic agents and/or modalities. The term administered “in combination,” as used herein, is understood to mean that two (or more) different treatments are delivered to the subject, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment improves an outcome to a greater extent, than would be seen if the second treatment were administered in the absence of the first treatment, or the analogous situation is seen with the first treatment. In certain embodiments, delivery is such that improvement of an outcome is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a disclosed method is administered in combination with an anticoagulant, for example, heparin, a citrate salt, etc. Anticoagulation protocols, such as systemic heparin or regional citrate, are currently established and routinely used in clinical hemofiltration. Additional exemplary anticoagulants include warfarin, FXa inhibitors (e.g., rivaroxaban, apixaban, betrixaban and edoxaban), thrombin inhibitors (e.g., hirudin, lepirudin, bivalirudin, argatroban and dabigatran), and coumarins.

Throughout the description, where devices or compositions are described as having, including, or comprising specific components, or where processes and methods are described as having, including, or comprising specific steps, it is contemplated that, additionally, there are devices or compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a device, a composition, or a method described herein can be combined in a variety of ways without departing from the spirit and scope of the present invention, whether explicit or implicit herein. For example, where reference is made to a particular device, that device can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of ”includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes the treatment of brain-dead organ donor subjects with a hemofilter prior to organ recovery and transplant.

No selection criteria were applied. Donors were selected as personnel and equipment were available. Donors were treated for 6 to 9 hours with a CLR 2.0 Hemofilter (Seastar Medical, Inc., Cardiff-by-the-Sea, Calif.). The filtrate-substitution fluid exchange dose was 35 ml/kg/hour. Substitution fluid was delivered pre-dilution (upstream of the hemofilter). Fluid removal was as indicated by the donor's hydration status. Vascular access was by a size 13.5 French scale (Fr) dialysis catheter in the right internal jugular vein, left internal jugular vein, and either femoral vein. Donors received no anticoagulation therapy.

Donor 1 was a 23-year-old male with a BMI of 21. Donor 1 experienced an opioid overdose. Donor 1 was found asystolic, CPR was initiated on scene, and circulation was restored in the ER, but Donor 1 did not recover consciousness. Anoxia was the cause of death. The P:F ratio for Donor 1 went from 167 to 392 following hemofiltration treatment. Seven organs were recovered from Donor 1 and four organs transplanted (two kidneys, a liver, and a heart).

Donor 2 was a 34-year-old male with a BMI of 22. Donor 2 experienced status epilepticus and cardiac arrest, with about 15 minutes downtime, and did not recover consciousness. Anoxia was the cause of death. The P:F ratio for Donor 2 went from 395 to 505 following hemofiltration treatment. 35 hours elapsed between hemofiltration treatment and organ recovery. Seven organs were recovered from Donor 2 and six organs transplanted (two kidneys, a liver, a heart, and two lungs).

Donor 3 was a 62-year-old male with a BMI of 31. Donor 3 was a 90-pack year smoker. As such, the lungs were not suitable for donation. Donor 3 experienced cardiac arrest at home, with greater than 30 minutes downtime. Anoxia was the cause of death. Donor 3 was oliganuric and listed at “liver only” for donor management. The P:F ratio for Donor 3 went from 80 to 67 following hemofiltration treatment. Three organs were ultimately recovered from Donor 3 and three organs transplanted (two kidneys and a liver). Notably, hemofiltration improved kidney function and allowed for transplant of both kidneys even though Donor 3 was oliganuric.

Donor 4 was a 61-year-old female with a BMI of 24. Donor 4 had hypertension (HTN) and a cerebrovascular accident (CVA). Donor 4 experienced intracerebral hemorrhage (ICH) and intravehicular hemorrhage (IVH), and did not recover consciousness. There was no arrest. CVA and stroke were the cause of death. The P:F ratio for Donor 4 went from 562 to 571 following hemofiltration treatment. Five organs were recovered from Donor 4 and four organs transplanted (a kidney, a liver, and two lungs).

Donor 5 was a 19-year-old male with a BMI of 28. Donor 5 had a gunshot wound to the head, which was also the cause of death. Donor 5 experienced refractory shock and progressed to multiple organ failure (MOF). There were multiple cardiac arrests and Donor 5 was declared medically unsuitable for organ donation. As a result, no organs were recovered from Donor 5 and no organs transplanted. The P:F ratio for Donor 5 went from 555 to >700 following hemofiltration treatment.

Donor 6 was a 53-year-old female with a BMI of 45. Donor 6 had an arrest. There was 40 minutes of post arrest CPR administered on the scene and in transit, and 17 minutes of CPR administered in the ER, where circulation returned. Donor 6 had pneumonia and pulmonary hypertension. Anoxia was the cause of death. The P:F ratio for Donor 6 went from 57 to 100 following hemofiltration treatment. Three organs were recovered from Donor 6 and one organ transplanted (a liver).

Donor 7 was a 40-year-old male with a BMI of 31. Donor 7 experienced an intracerebral hemorrhage (ICH) and stroke. CVA/stroke was the cause of death. The P:F ratio for Donor 7 went from 537 to 528 following hemofiltration treatment. Six organs were recovered from Donor 7 and five organs transplanted (two kidneys, a liver, and two lungs).

On average, 4.8 organs were recovered per donor, approximately 1 additional organ per donor relative to the historic average. Additionally, on average, 3.8 organs were transplanted per donor, approximately 0.5 additional organs per donor relative to the historic average.

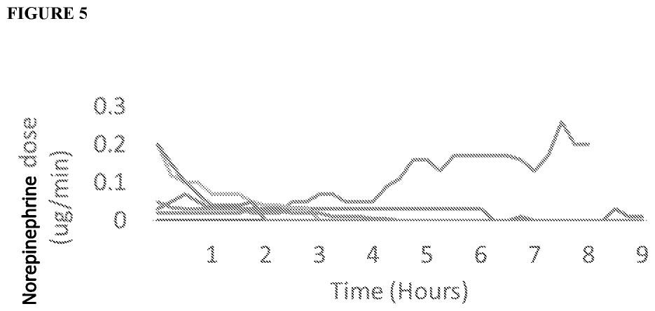

The dose of a vasopressor, norepinephrine, was reduced in most donors within two hours, as shown in FIG. 5. These results demonstrate that hemofiltration reduced the need for vasopressor administration in donors, one factor considered in donor management guidelines.

Average serum chemistry for the seven donors before hemofiltration treatment (“pre”), after hemofiltration treatment (“post”) and prior to organ recovery (“pre-OR”) is shown in TABLE 2. These results demonstrate that hemofiltration did not markedly alter donor serum chemistry, suggesting that treatment according to the invention does not adversely affect blood levels of solutes necessary for health of the donor.

TABLE 2

| Analyte | Pre | Post | Pre-OR |
|---|---|---|---|
| Na | 144 | 141 | 144 |
| K | 4.4 | 4.8 | 4.3 |
| Cl | 111 | 108 | 108 |
| CO₂ | 24 | 22 | 23 |
| BUN | 19 | 20 | 28 |
| Cr | 1.4 | 1.3 | 1.5 |
| Gluc | 145 | 187 | 221 |
| Alb | 2.5 | 3.0 | 3.0 |
| T Prot | 5.6 | 6.4 | 6.5 |
| Ca | 8.4 | 8.5 | 8.5 |
| iCa | 1.22 | 1.15 | 1.23 |
| Mg | 2.0 | 2.0 | 2.3 |
| PO₄ | 3.13 | 3.88 | 3.70 |
| T. Bili | 0.4 | 0.8 | 0.7 |
| D. Bili | 0.2 | 0.5 | 0.2 |
| AST | 62 | 61 | 37 |
| ALT | 77 | 78 | 119 |

As of 5.8 to 8.6 months post-transplant, 23/23 organ recipients were alive and 23/23 grafts were functioning.

Together, these results demonstrate that treatment of brain-dead organ donors with a hemofilter, e.g., a CLR 2.0 hemofilter, improves organ recovery and transplant outcomes.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of preparing one or more organs from a donor for transplantation to a recipient, the method comprising contacting blood from the donor with an extracorporeal membrane defining a plurality of pores having an average pore size of at least 60 kDa to permit proinflammatory molecules in the blood to pass through the pores for removal from the blood, whereupon the blood depleted of the proinflammatory molecules is returned to the donor.

2. The method of claim 1, wherein the blood from the donor is contacted with the extracorporeal membrane for at least 30 minutes prior to procuring the organ or organs from the donor.

3. The method of claim 1 or 2, wherein the pores are defined by a wall of a semi-permeable hollow fiber.

4. A method of preparing one or more organs from a donor for transplantation to a recipient, the method comprising passing blood from the donor through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, proinflammatory molecules from the blood pass through the pores and are removed from the blood, whereupon the blood depleted of proinflammatory molecules is returned to the donor, wherein the blood from the donor is passed through the cartridge for at least 30 minutes prior to procuring the organ or organs.

5. The method of any one of claims 1-4, wherein the pores have an average pore size of from about 60 kDa to about 150 kDa.

6. The method of any one of claims 1-4, wherein the pores have an average pore size of greater than 65 kDa.

7. The method of any one of claims 1-4 wherein the pores have an average pore size of no greater than 65 kDa.

8. The method of any one of claims 1-4, wherein the pores have an average pore size from about 60 kDa to about 65 kDa.

9. The method of any one of claims 1-8, wherein the donor is a brain-dead donor.

10. The method of any one of claims 1-9, wherein the donor is human.

11. The method of any one of claims 1-10, wherein the donor is an adult human donor.

12. The method of any one of claims 1-10, wherein the donor is a pediatric human donor.

13. The method of any of claims 1-12, wherein the organ or organs is one or more of a heart, lungs, kidney, liver, intestine, or pancreas.

14. The method of any one of claims 3-13, wherein the hollow fibers comprise a polymer.

15. The method of any one of claims 3-13, wherein the hollow fibers comprise polysulfone.

16. The method of any one of claims 3-11, wherein the lumen of the hollow fibers has a diameter from about 100 μM to about 700 μM.

17. The method of claim 16, wherein the lumen has a diameter from about 175 μM to about 225 μM.

18. The method of claim 16, wherein the lumen has a diameter from about 600 μM to about 700 μM.

19. The method of any one of claims 3-18, wherein the surface area of the hollow fibers is from about 0.01 m² to about 4.0 m².

20. The method of claim 19, wherein the surface area of the hollow fibers is from about 1.9 m² to about 2.1 m².

21. The method of claim 19, wherein the surface area of the hollow fibers is from about 0.05 m²to about 0.1 m², or from about 0.25 m² to about 0.75 m², or from about 1.0 m² to about 1.5 m².

22. The method of any of claims 4-21, wherein the cartridge comprises a fluid inlet port and a fluid outlet port.

23. The method of any of claims 4-22, wherein the cartridge comprises one or more ultrafiltrate ports.

24. The method of any one of claims 1-23, wherein the proinflammatory molecules removed from the blood are selected from one or more of IL-6, IL-8, TNF-α, IL-1β, MCP-1, CCL2, IP-10, CXCL10, C3a, C5a, soluble TNF receptor II, matrix metalloproteinase-9, IL-10, soluble gp130, lipopolysaccharide (LPS) or procalcitonin.

25. The method of any one of claims 4-24 wherein the flow rate of blood through the cartridge is from about 100 mL/min to about 600 mL/min.

26. The method of any one of clams 4-24, wherein the flow rate of blood through the cartridge is from about 100 mL/min to about 400 mL/min.

27. The method of any one of claims 4-24, wherein the flow rate of blood through the cartridge is from about 150 mL/min to about 250 mL/min.

28. The method of any one of claims 1-27, wherein the donor's blood is passed through the cartridge or contacted with the membrane for at least 30 minutes to about 120 hours prior to procuring one or more organs from the donor.

29. The method of any one of claims 1-27, wherein the donor's blood is passed through the cartridge or contacted with the membrane for about 1 hour to about 72 hours prior to procuring one or more organs from the donor.

30. The method of any one of claims 1-27, wherein the donor's blood is passed through the cartridge or contacted with the membrane for about 1 hour to about 24 hours prior to procuring one or more organs from the donor.

31. The method of any one of claims 1-27, wherein the donor's blood is passed through the cartridge or contacted with the membrane for a time period selected from about 1 hour, from about 3 hours to about 6 hours, from about 6 hours to about 12 hours, about 24 hours, about 48 hours or about 72 hours prior to procuring one or more organs from the donor.

32. The method of any one of claims 1-31, wherein after the donor's blood is passed through the cartridge or contacted with the membrane, the donor shows an improvement in one or more of mean arterial pressure, central venous pressure, ejection fraction, arterial blood gas, partial pressure of arterial oxygen to fraction of inspired oxygen ratio (PaO_{2:}FiO₂), serum sodium, urine output, glucose level, hemoglobin level, or reduction in dose or number of vasopressors required to maintain blood pressure.

33. The method of any one of claims 1-32, wherein the cartridge or membrane is connected to the donor via an extracorporeal circuit comprising a line from an artery or vein of the donor, a line to a vein of the donor, and an ultrafiltrate collection container.

34. The method of claim 33, wherein the extracorporeal circulation system further comprises one or more of an ultrafiltrate pump, ultrafiltrate pressure sensor, blood sensor, filter pressure sensor, venous pressure sensor, access pressure sensor, IV fluid return pump, or a temperature regulator.

35. The method of any one of claims 4-34, wherein the ultrafiltration rate of the cartridge is from about 40 mL/min to about 180 mL/min.

36. The method of any one of claims 1-35, wherein the method maintains or revitalizes one or more organs of the donor so that the one or more organs is suitable for transplantation to a recipient.

37. The method of any one of claims 1-36, further comprising the step of procuring one or more organs from the donor.

38. The method of any one of claims 1-37, wherein the membrane comprises a polymer.
